(19) United States
(12) Patent Application Publication
(10) Pub. No.: US 2007/0041493 A1
(43) Pub. Date: Feb. 22, 2007

(54) Method and apparatus for producing an intensity modulated beam of radiation

(76) Inventors: Steve Webb, London (GB); Wolfgang Schlegel, Wiesenback (DE); Gernot Echner, Wiesenback (DE); Gunther Hartmann, Heidelberg (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/555,916

(22) PCT Filed: May 10, 2004
(86) PCT No.: PCT/GB04/02009

§ 371 Date: Jul. 24, 2006

(30) Foreign Application Priority Data
May 8, 2003 (GB) 0310596.2

(51) Int. Cl. A61N 5/10; G21K 1/02

(52) U.S. Cl. 378/65; 378/147

(57) ABSTRACT

A method of intensity-modulating a beam of radiation from a radiation source is provided, the method including the steps of: a) providing a collimator having a window which allows the passage of radiation from the radiation source and which defines when viewed from the radiation source a two-dimensional array of bixels, the collimator further having a plurality of independently movable radiation attenuators which are constrained to move along columns or rows of the array to block selected bixels thereof, whereby, within each column or row along which the attenuators are movable, at least one arrangement of the respective attenuator(s) results in a pair of open bixels sandwiching a blocked bixel or line of blocked bixels; b) positioning the collimator in the path of the radiation source; and c) repeatedly: positioning the radiation attenuators within the window to form at each repeat a different pattern of bixels; and irradiating the collimator, until a predetermined pattern of radiation intensities for the beam of radiation has been delivered through the collimator. A corresponding apparatus for producing an intensity-modulated beam of radiation which is operable in accordance with the above method, and a method of radiotherapy treatment performed on a human or animal body comprising subjecting the body to an intensity-modulated beam of radiation formed using the above method are also provided.

The present invention relates to methods and apparatuses for delivering an intensity modulated beam (IMB) of radiation. It is particularly, but not exclusively concerned with methods of delivering such a beam without using a multi-leaf collimator (MLC), and also with intensity-modulated radiotherapy (IMRT).

IMRT is a well established technique of improving the conformality of dose distributions thereby sparing normal tissues from radiation damage. The most commonly used present techniques deliver IMRT using linear accelerators (“linacs”) fitted with one of several types of MLC. In particular the clinical implementation of IMRT has been dominated by the step-and-shoot or multiple-static-field technique, the dynamic MLC (DMLC) technique and the NOMOS MIMiC (Multivane Intensity Modulating Collimator). See e.g. U.S. Pat. No. 5,317,616 and U.S. Pat. No. 5,351,280.

However, not all IMRT centres have linacs fitted with an MLC. Centres with a non-MLC linac or which deliver radiotherapy using a cobalt-60 machine (which cannot use an MLC) would benefit from alternative techniques to deliver IMRT. This is particularly useful in countries where the use of linacs is less common.

The first attempts to address this were by Dai and Hu (1999 Medical Physics 26 (12) 2562-2570) who questioned whether IMRT could be delivered by a “step-and-shoot” technique using just the jaws of a radiation delivery machine. They developed detailed decomposition schemes for what has become known as the “jaws-only” (JO) IMRT technique. They concluded that, whilst this inevitably required more field components and more monitor units (MUs) than the use of an MLC, the technique was nevertheless nearly feasible. The JO decomposition of a large number of random square matrices of modulation (IMBs) with several matrix sizes N×N and with varying peak value Iₚ was studied.

The present inventor extended this idea by combining the concept of variable jaw positions with the concept of a variable beam mask (Webb S, 2002, Phys. Med. Biol. 47 257-275; 1869-1879; N217-222). This mask would have bixel(beam-element)-size apertures which were either open (e.g. air) or closed (e.g. tungsten). Variable effective patterns of the mask can be obtained by selecting different cut-outs from a large area mask with variable jaw positions and a variable mask position. By judicious choices of jaw positions combined with mask positions, the “jaws-plus-mask” (J+M) technique is able to provide a huge number of different modulation patterns.

In order to study the potential of this idea, a tool was developed to quantitatively analyse the effort that is required to decompose a given square matrix of modulation with various IMRT techniques. This tool is based on a stripping algorithm which removes in a step-by-step manner sub-patterns of modulations which can be jointly provided from the pattern remaining from the previous step. The algorithm is then applied to a large number of randomly generated starting patterns. The tool finally yields statistical outcomes for decomposing random IMBs of varying size N and peak value Iₚ. Whilst practical radiotherapy IMBs are generally not entirely random, the analysis of random square matrices of modulation has become a standard way to benchmark IMRT delivery techniques.

Three types of mask for the J+M technique were studied. The first two comprised either a regular or random pattern of open apertures arranged in a single plate and capable of movement in just the two orthogonal directions of the jaw movement. The third comprised a number of parked single-bixel attenuators (SBAs) which could be brought into the jaws-collimated field components to block some bixels. It was found that the number of components and number of MUs required for a J+M decomposition was always less than for the corresponding JO decomposition since the component irradiations using a mask can group isolated bixels which would otherwise have to be individually irradiated. These studies also showed that a modulation-splitting technique could also greatly reduce the statistically average number of field components (but not the number of MUs).

All these developments address the worst-case scenario of highly modulated IMBs, e.g. those created by the NOMOS CORVUS planning system. Several recent developments have aimed to introduce smoothing constraints into the inverse planning to generate smoother IMBs which do not over-sacrifice dose conformality. It follows logically then that any IMRT delivery technique that copes with highly-modulated IMBs such as the benchmarks, will cope better with less highly modulated IMBs.

One observation emerging from the J+M decomposition studies is that the significant reduction of the number of field components is a direct consequence of the ability to couple together otherwise isolated islands of fluence and deliver them together (something the use of jaws only technique cannot achieve). Moreover, it was noticed that most of the couplings link bixels which are quite close together in space in the IMB. This is a consequence of the design of the mask. For the single-plate masks it becomes increasingly likely that, as the decomposition nears its close for any particular IMB, the remaining isolated bixels will be quite widely spaced out in the residual IMB. Hence, given the structure of the proposed masks, there is no mechanism to couple these bixels.

Whilst the J+M technique using relocatable SBAs produced good theoretical results, the implementation of such an arrangement presents a number of practical and engineering difficulties. In particular, consideration has to be given to how the SBAs are moved into and between their positions in the collimator, and how they are retained in those positions during delivery of the radiation. This is particularly relevant when considering the delivery of radiation at angles other than the vertical.

The delivery of IMBs has also been addressed by Swerdloff et al. in U.S. Pat. No. 5,317,61 and U.S. Pat. No. 5,351,280. These documents discuss use of a linear or “slit” device with removable radiation attenuating leaves which adjust a beam of adjacent rays dispersed within a single beam plane. This apparatus is used in the NOMOS MIMiC. However, a slit device of this type suffers from the fact that it needs to be frequently relocated in order to deliver the complete beam, and as a result requires close attention to be paid to the matching lines to ensure that the patient's position relative to the collimator is maintained throughout delivery.

The present invention seeks to provide a method and apparatus for producing an intensity modulated beam of radiation with comparable performance to the jaws and mask technique with improved practical implementations.

At its most general, the present invention provides a method of intensity-modulating a beam of radiation from a radiation source, the method including the steps of:
a) providing a collimator having a window which allows the passage of radiation from the radiation source and which defines when viewed from the radiation source a two-dimensional array of bixels, the collimator further having a plurality of independently movable radiation attenuators which are constrained to move along columns or rows of the array to block selected bixels thereof;
b) positioning the collimator in the path of the radiation source; and
c) repeatedly:
   positioning the radiation attenuators within the window to form at each repeat a different pattern of bixels, and
   irradiating the collimator;
      until a predetermined pattern of radiation intensities for the beam of radiation has been delivered through the collimator.

According to a first aspect of the present invention there is provided a method of intensity-modulating a beam of radiation from a radiation source, the method including the steps of:
a) providing a collimator having a window which allows the passage of radiation from the radiation source and which defines when viewed from the radiation source a two-dimensional array of bixels, the collimator further having a plurality of independently movable radiation attenuators which are constrained to move along columns or rows of the array to block selected bixels thereof, whereby, within each column or row along which the attenuators are movable, at least one arrangement of the respective attenuator(s) results in a pair of open bixels sandwiching a blocked bixel or line of blocked bixels;
b) positioning the collimator in the path of the radiation source; and
c) repeatedly:
   positioning the radiation attenuators within the window to form at each repeat a different pattern of bixels, and
   irradiating the collimator;
      until a predetermined pattern of radiation intensities for the beam of radiation has been delivered through the collimator.

The collimator may be repositioned one or more times during the performance of step c).

By using this method, an intensity modulated beam having a predetermined pattern of radiation intensities can be built up from a number of component irradiations. The movable radiation attenuators in the collimator window allow a variety of different collimator aperture patterns to be created and used. Since the radiation attenuators are independently movable, many different aperture patterns can be formed from a relatively small number of attenuators. By “independently movable”, we preferably mean that each attenuator can be moved without moving any of the other attenuators.

Constraining the attenuators to move along columns or rows of the array allows them to be positioned and relocated between each irradiation using simple mechanical arrangements, for example using position controlling rods attached to each attenuator. Preferably, each attenuator is movable along only one row or column. Furthermore, all the attenuators may only be movable along respective columns (or all the attenuators may only be movable along respective rows). For example, a bixel array formed from N adjacent columns of bixels can be patterned using N attenuators, each attenuator being only moveable along a respective column.

However, more than one attenuator can share a column or row. For example, the bixel array with N adjacent columns of bixels can be patterned using 2N attenuators, each column having two independently moveable attenuators.

To reduce the number of irradiations necessary to deliver the predetermined pattern of radiation intensities, the size and/or position of the window may be adjusted one or more times during the performance of step c). Size adjustment corresponds to changes in the number of rows and/or the number of columns in the array of bixels, whereas position adjustment corresponds to a displacement of the window. Typically the collimator has jaws which define the boundary of the window, and the adjustment can be accomplished by moving the appropriate jaw or jaws.

An additional or alternative way of reducing the number of irradiations needed to deliver the predetermined pattern of intensities is to rotate the collimator about an axis perpendicular to the window one or more times during the performance of step c). This may allow a different set of collimator apertures to be created.

Each attenuator may attenuate a single bixel.

Indeed, a second aspect of the present invention provides a method of intensity-modulating a beam of radiation from a radiation source, the method including the steps of:
a) providing a collimator having a window which allows the passage of radiation from the radiation source and which defines when viewed from the radiation source a two-dimensional array of bixels, the collimator further having a plurality of independently movable radiation attenuators which are constrained to move along columns or rows of the array to selectively block selected only single bixels thereof;
b) positioning the collimator in the path of the radiation source; and
c) repeatedly:
   positioning the radiation attenuators within the window to form at each repeat a different pattern of bixels; and
   irradiating the collimator;
      until a predetermined pattern of radiation intensities for the beam of radiation has been delivered through the collimator.

The preferred and optional features discussed above in relation to the first aspect may also be incorporated in the method of this aspect.

A third aspect of the present invention provides a method of intensity-modulating a beam of radiation from a radiation source, the method including the steps of:
a) providing a collimator having a window which allows the passage of radiation from the radiation source and which defines when viewed from the radiation source a two-dimensional array of bixels, the collimator further having a plurality of independently movable radiation attenuators which are constrained to move along columns or rows of the array to block selected bixels thereof, whereby movement of any one of said attenuators from a first position to a second position causes opening of a bixel of the first position and blocking of a bixel of the second position;
b) positioning the collimator in the path of the radiation source; and
c) repeatedly:
   positioning the radiation attenuators within the window to form at each repeat a different pattern of bixels; and
   irradiating the collimator;
      until a predetermined pattern of radiation intensities for the beam of radiation has been delivered through the collimator.

The preferred and optional features discussed above in relation to the first aspect may also be incorporated in the method of this aspect.

A further aspect of the invention provides a method of radiotherapy treatment performed on a human or animal body comprising subjecting the body to an intensity-modulated beam of radiation formed by the method of any of the above aspects of the invention.

The method of treatment may also include one or more of the further steps of: determining the pattern of radiation intensities to be delivered; positioning the patient and/or a radiation source and the collimator to deliver the intensity modulated beam to a particular part of the patient's anatomy.

A method of treatment according to this aspect of the present invention provides a relatively simple and practical way of delivering an IMB to a patient without the need for a multi-leaf collimator (MLC).

In general terms, the apparatus of the present invention is an apparatus for producing an intensity-modulated beam of radiation which is operable in accordance with the method of any one of the above method aspects of the invention, the apparatus including:
a radiation source;
a collimator having a window which allows the passage of radiation from the radiation source and defines when viewed from the radiation source a two-dimensional array of bixels, the collimator further having a plurality of independently movable radiation attenuators which are constrained to move along columns or rows of the array to block selected bixels thereof, whereby, within each column, at least one arrangement of the respective attenuator(s) results in a pair of open bixels sandwiching a blocked bixel or line of blocked bixels; and
a control system for controlling the position of the radiation attenuators within the window of the collimator.

A further aspect of the present invention provides an apparatus for producing an intensity-modulated beam of radiation which is operable in accordance with the method of the first aspect of the invention, the apparatus including:
a radiation source;
a collimator having a window which allows the passage of radiation from the radiation source and defines when viewed from the radiation source a two-dimensional array of bixels, the collimator further having a plurality of independently movable radiation attenuators which are constrained to move along columns or rows of the array to block selected bixels thereof, whereby, within each column or row along which the attenuators are movable, at least one arrangement of the respective attenuator(s) results in a pair of open bixels sandwiching a blocked bixel or line of blocked bixels; and
a control system for controlling the position of the radiation attenuators within the window of the collimator.

The movable radiation attenuators in the collimator window can be positioned and relocated between each irradiation, for example using position controlling rods attached to each attenuator which may be driven by solenoids, pneumatics, hydraulics, electric drive units or other means.

To reduce the number of irradiations necessary to deliver a predetermined pattern of radiation intensities, the size of the collimator window and/or position of the collimator window relative to the radiation source may be adjustable, in which case the control system may also control the size and/or position of the collimator window.

Preferably, each attenuator is movable along only one row or column. Furthermore, all the attenuators may only be movable along respective columns (or all the attenuators may only be movable along respective rows). The attenuators may therefore move parallel to each other. Preferably there are at least two attenuators for each column or row of the array. Each attenuator may attenuate a single bixel.

An additional or alternative way of reducing the number of irradiations needed to deliver the predetermined pattern of intensities is if the collimator is able to rotate about a axis perpendicular to the window. This may allow a different set of collimator apertures to be created. Preferably, the control system also controls the rotation of the collimator.

The collimator is preferably mounted on a double-cradle arrangement so that when moved the collimator is always maintained at substantially the same distance from the radiation source. In this way, the bixels of the window array can be kept at the same size. They can also be focussed to the source so the penumbra formed by each attenuator is constant.

The attenuators may be tapered in the direction of the beam to ensure that they block a single bixel of radiation along their entire length without attenuating the radiation in neighbouring bixels.

The control system can be configured to implement step c) of the method. The control system may also control the intensity of the beam emitted from the radiation source.

Thus the control system may operate according to a predetermined set of co-ordinates each of which contains details of one or more of: the position of the attenuators in the window; the position of the collimator relative to the radiation source; the size or dimensions of the collimator window; and the intensity of radiation to be delivered.

A further aspect of the present invention provides an apparatus for producing an intensity-modulated beam of radiation which is operable in accordance with the method of the second aspect of the invention, the apparatus including:
a radiation source;
a collimator having a window which allows the passage of radiation from the radiation source and defines when viewed from the radiation source a two-dimensional array of bixels, the collimator further having a plurality of independently movable radiation attenuators which are constrained to move along columns or rows of the array to selectively block selected only single bixels thereof; and
a control system for controlling the position of the radiation attenuators within the window of the collimator.

The preferred and optional features discussed above in relation to the previous aspect may also be incorporated in the apparatus of this aspect.

A further aspect of the present invention provides an apparatus for producing an intensity-modulated beam of radiation which is operable in accordance with the method of the third aspect of the invention, the apparatus including:
a radiation source;
a collimator having a window which allows the passage of radiation from the radiation source and defines when viewed from the radiation source a two-dimensional array of bixels, the collimator further having a plurality of independently movable radiation attenuators which are constrained to move along columns or rows of the array to block selected bixels thereof, whereby movement of any one of said attenuators from a first position to a second position causes opening of a bixel of the first position and blocking of a bixel of the second position; and
a control system for controlling the position of the radiation attenuators within the window of the collimator.

The preferred and optional features discussed above in relation to the first of the apparatus aspects may also be incorporated in the apparatus of this aspect.

According to a further aspect of the present invention, there is provided the collimator of any of the apparatuses of the previous aspects of the invention. Such a collimator can be used in method and apparatus of the previous aspects.

Embodiments of the invention will now be illustrated with reference to the accompanying drawings, in which:

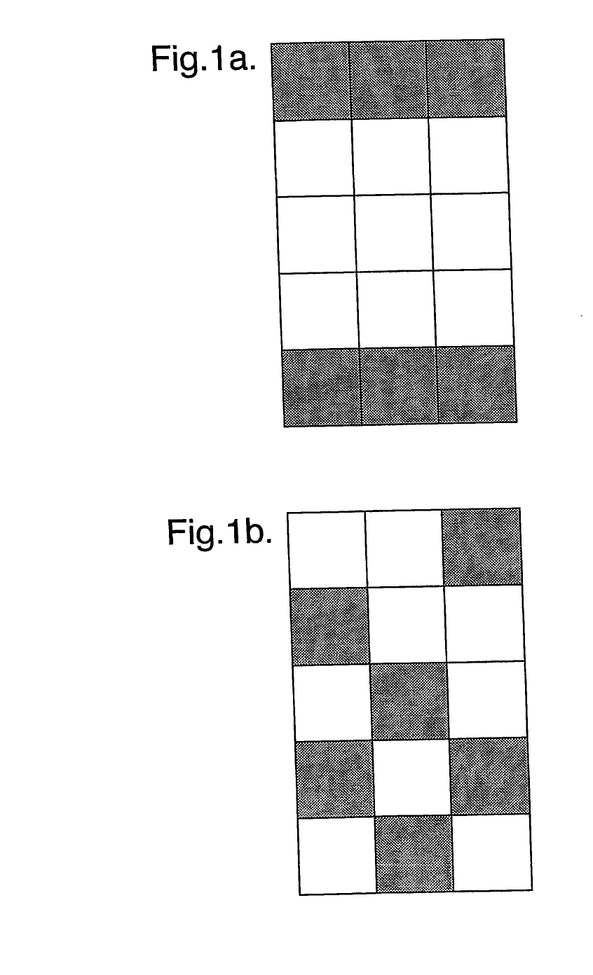

FIGS. 1a and 1b show a variable aperture collimator (VApC) according to a first embodiment of the present invention in the “park” position, and in one of the aperture positions;

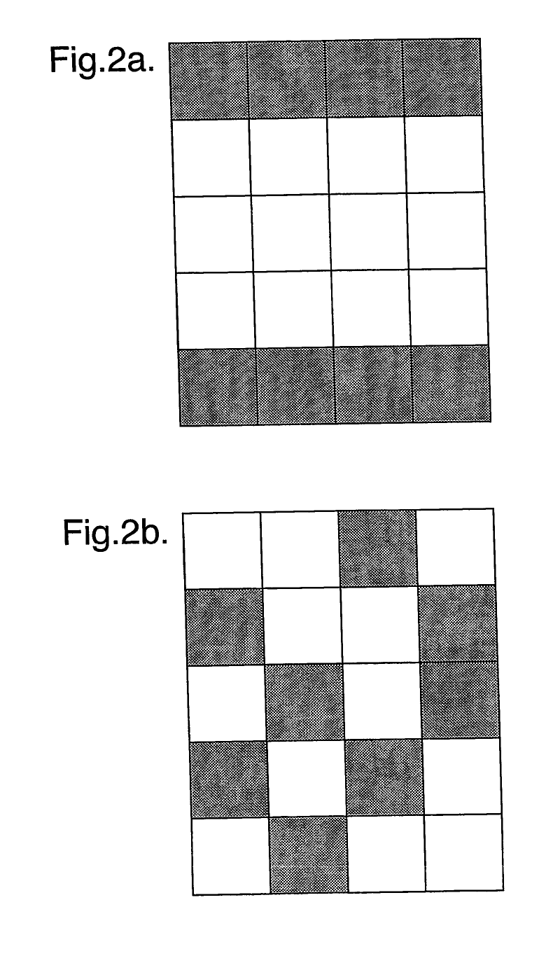

FIGS. 2a and 2b show a VApC according to a second embodiment of the present invention in the “park” position, and in one of the aperture positions;

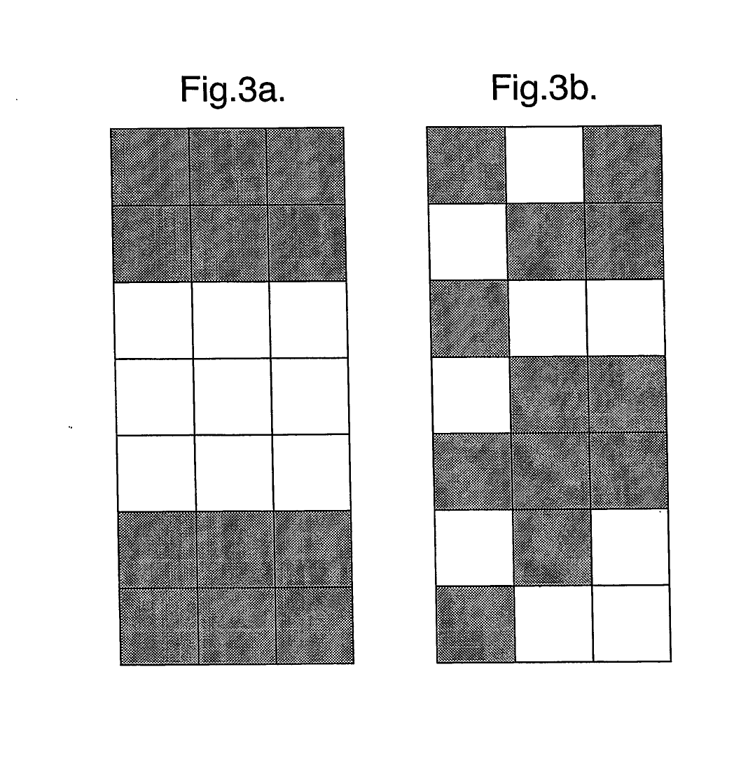

FIGS. 3a and 3b show a VApC according to a third embodiment of the present invention in the “park” position, and in one of the aperture positions;

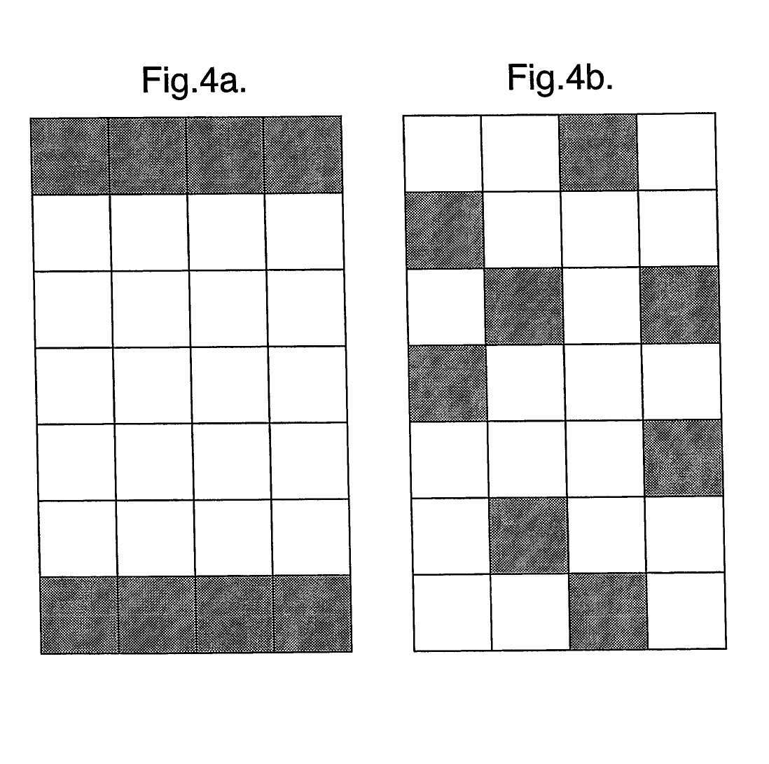

FIGS. 4a and 4b show a VApC according to a fourth embodiment of the present invention in the “park” position, and in one of the aperture positions;

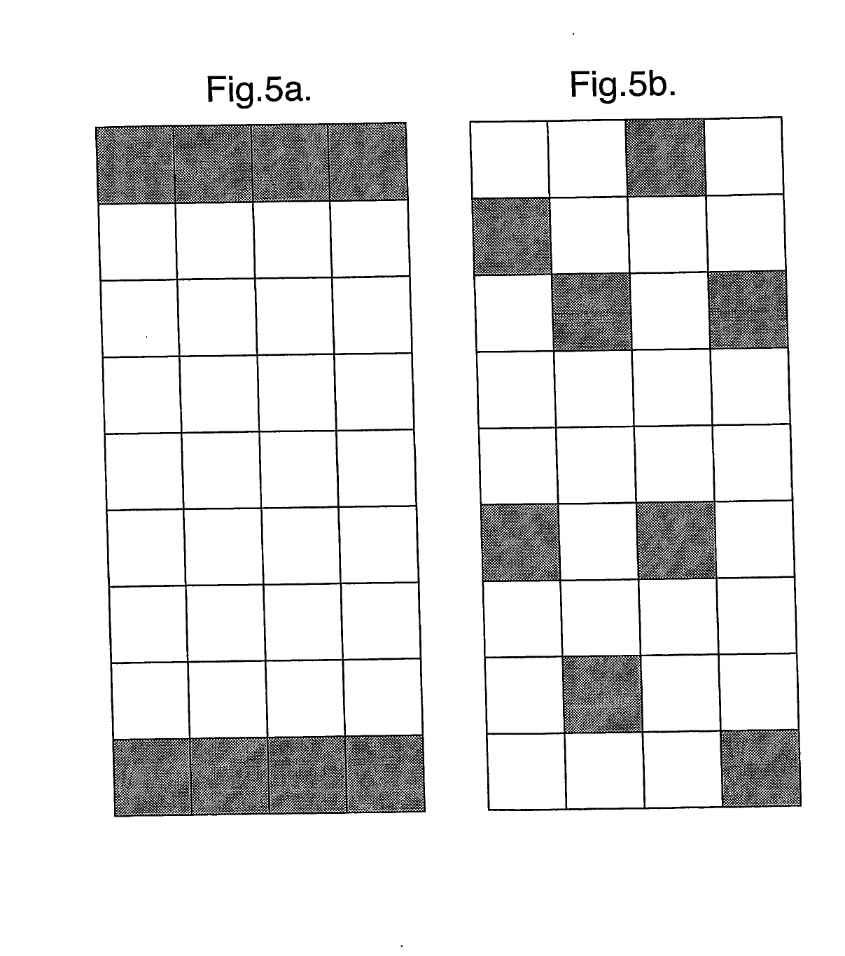

FIGS. 5a and 5b show a VApC according to a fifth embodiment of the present invention in the “park” position, and in one of the aperture positions;

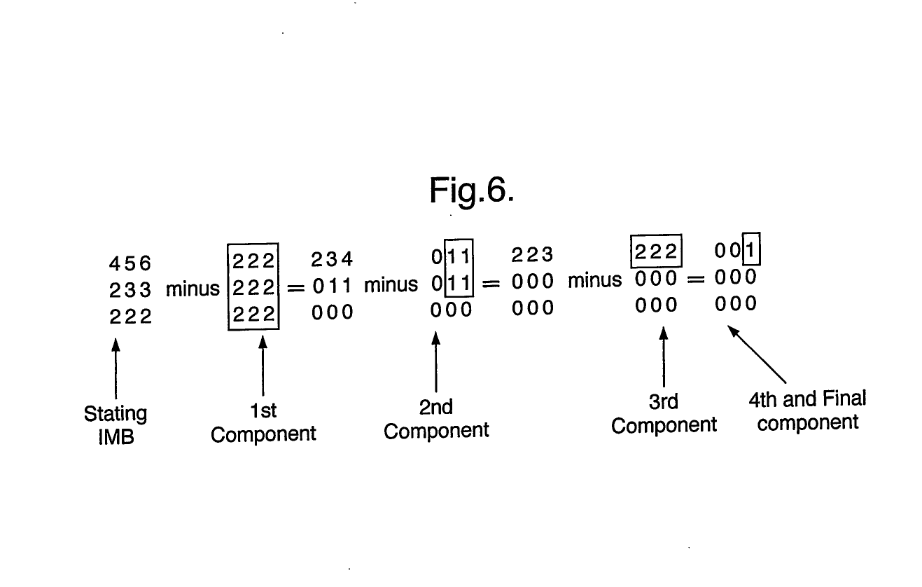

FIG. 6 shows a simplified example of a stripping algorithm using the jaws-only technique;

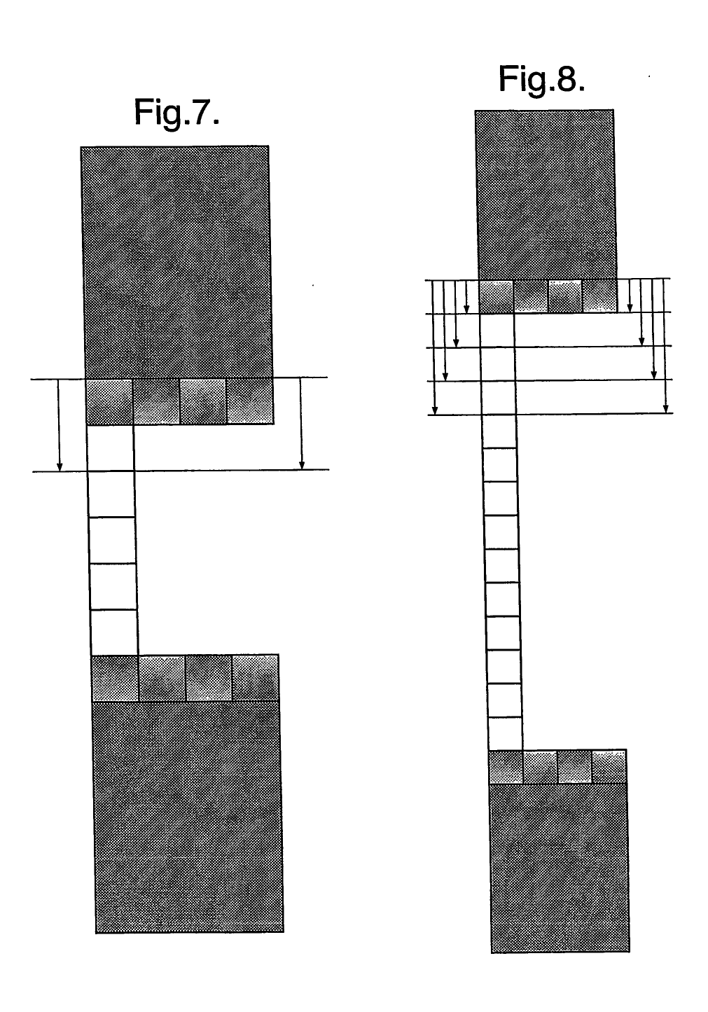

FIG. 7 shows a “hybrid” VApC according to a further embodiment of the present invention in which the aperture size can be changed from a VApC according to the fourth embodiment to a VApC according to the second embodiment;

FIG. 8 shows another “hybrid” VApC according to a further embodiment of the present invention in which all possible sub-combinations of aperture size are included;

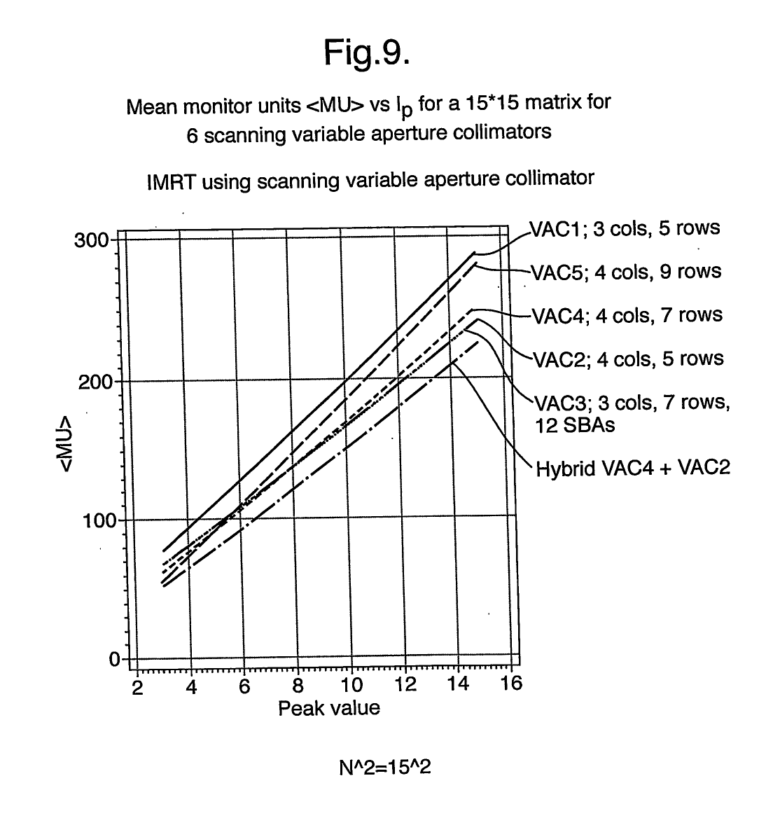

FIGS. 9 to 11 show the results of testing different VApCs using a benchmark of 1000 15×15 IMBs with fluences of individual bixels being randomly selected between 3 and Iₚ, these Figures showing either the mean number of components <M> required to decompose an IMB, or the mean number of monitor units <MU> required to decompose an IMB for each value of Iₚ;

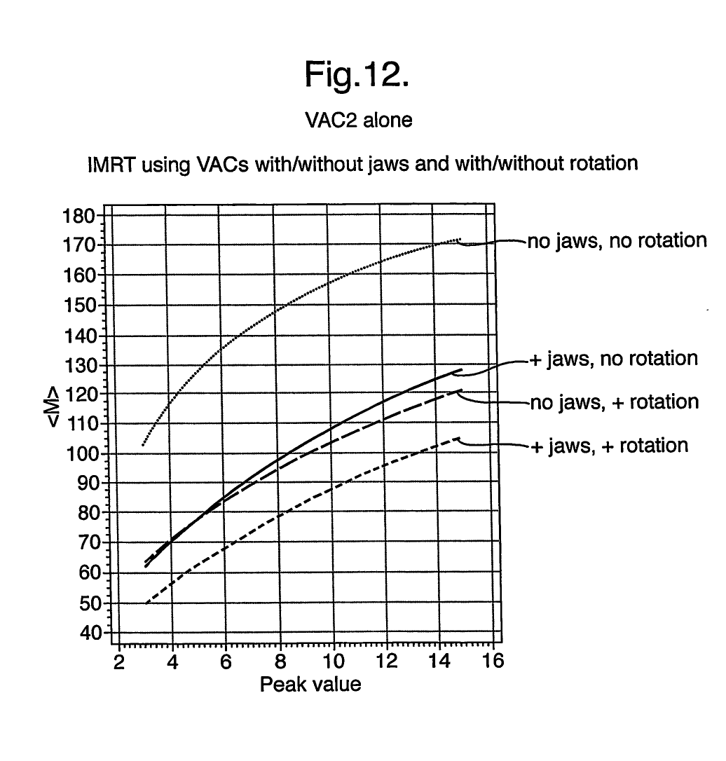

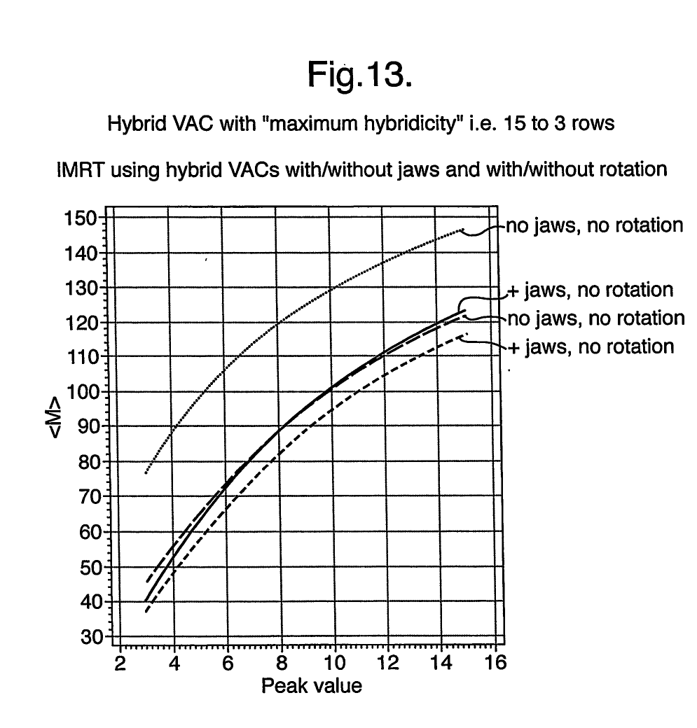

FIGS. 12 and 13 show a comparison of the mean number of components <M> required to decompose the benchmark IMBs using the VApC2 and a hybrid VApC with 15 to 3 rows depending on whether the jaws are used to reduce the number of columns, the VApC can be rotated, or both;

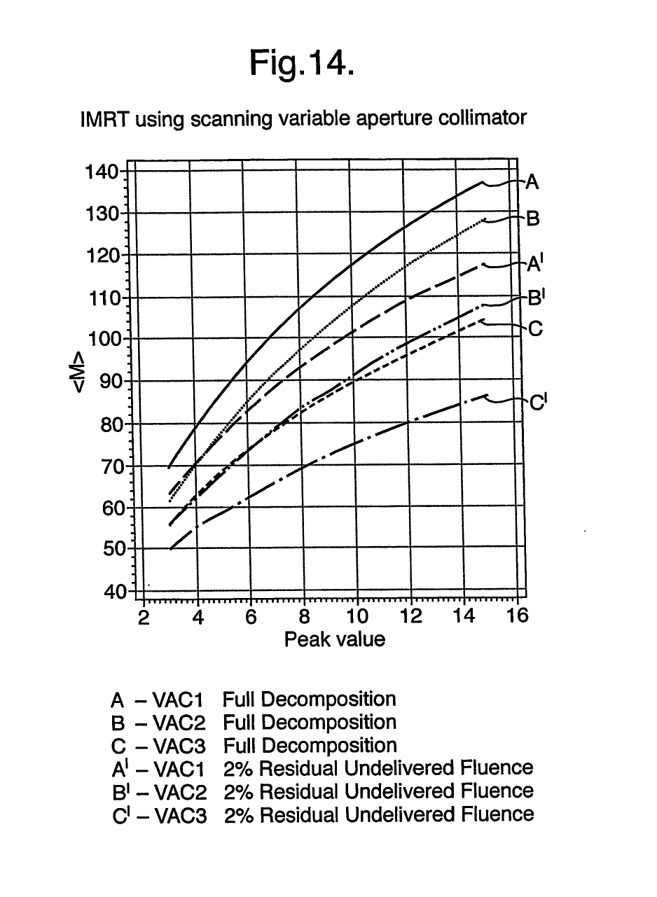

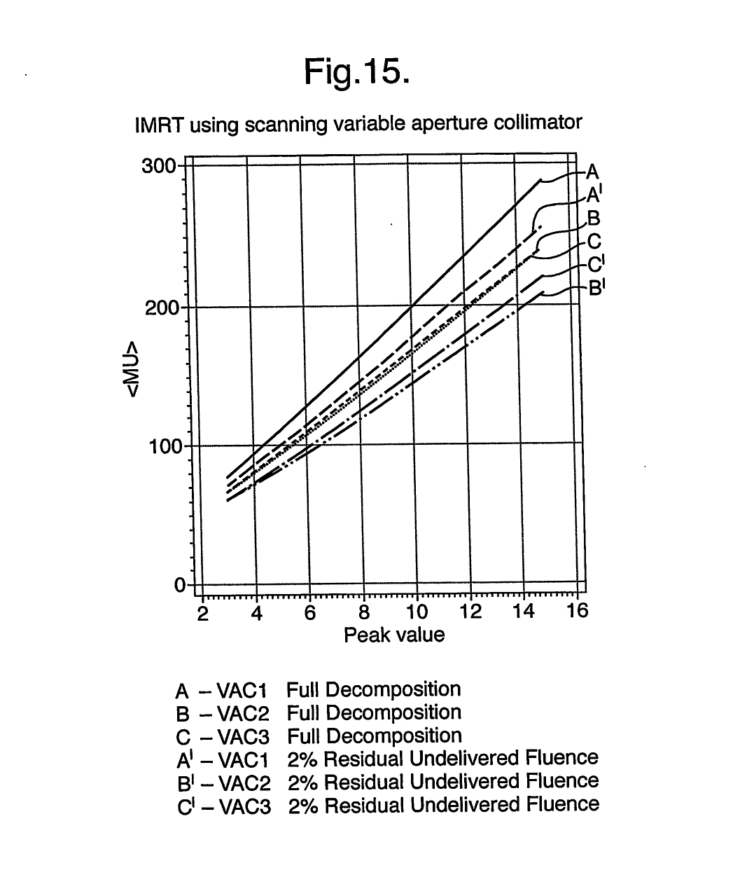

FIGS. 14 and 15 show a comparison of the mean number of components <M> required to decompose an IMB, and the mean number of monitor units <MU> required to decompose an IMB for each value of Iₚ between using complete decomposition and when the decomposition is stopped with 2% residual fluence remaining;

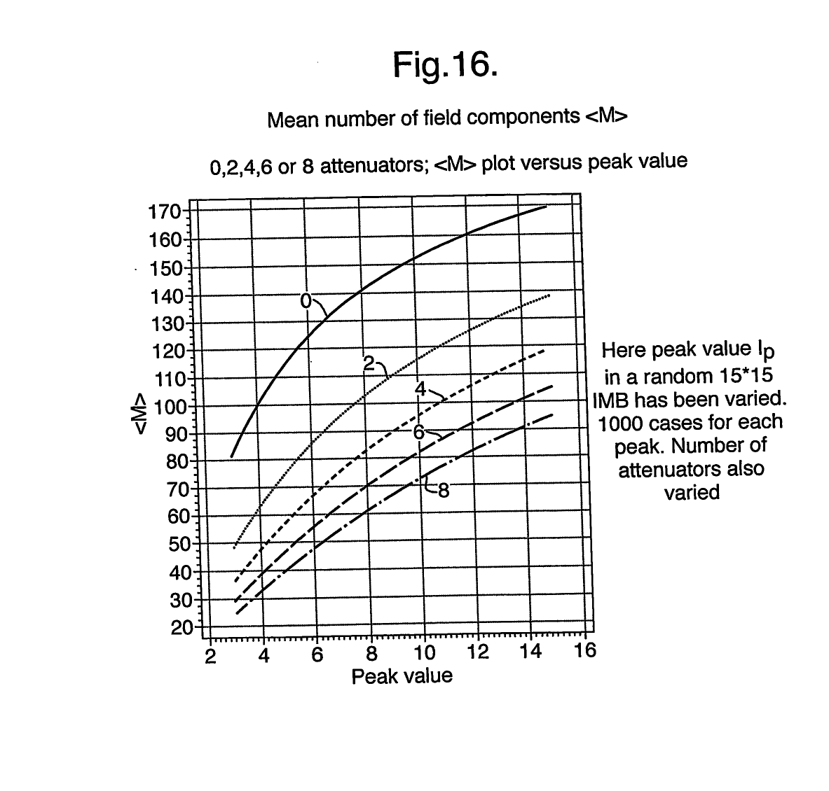

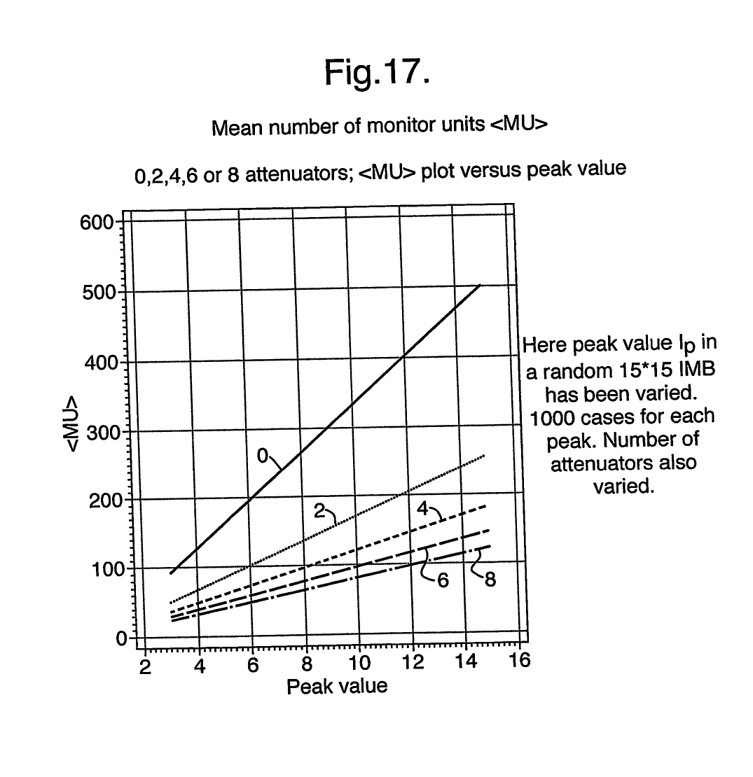

FIGS. 16 and 17 show the mean number of components <M> required to decompose an IMB, and the mean number of monitor units <MU> required to decompose an IMB for each value of Iₚ using the prior art techniques of jaws and mask (J+M) with the stated number of individually relocatable single-bixel attenuators (zero attenuators represents the jaws only (JO) technique), using the same benchmark as FIGS. 9 to 15 above, for comparison;

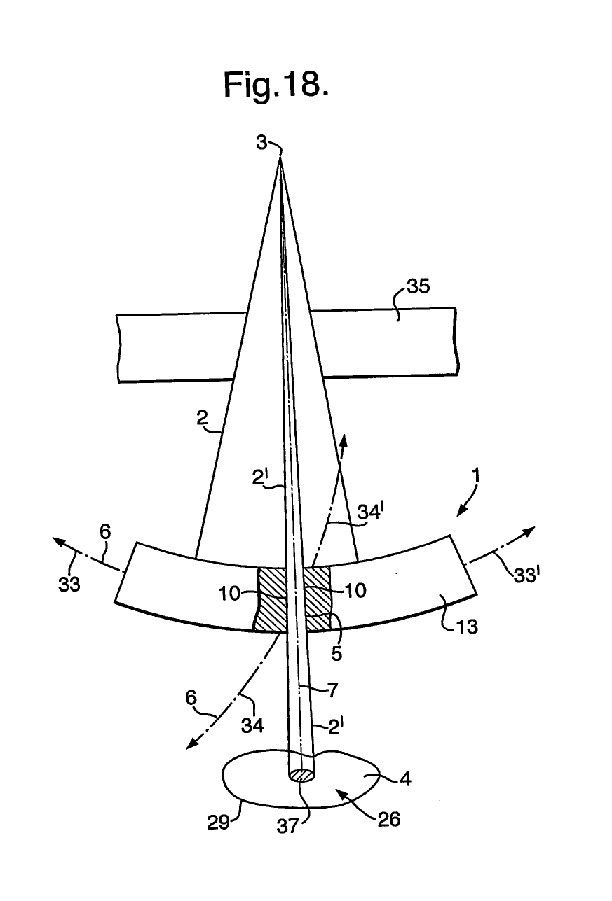

FIG. 18 shows the underlying principle of the type of apparatus in which a VApC according to an embodiment of the present invention may be used;

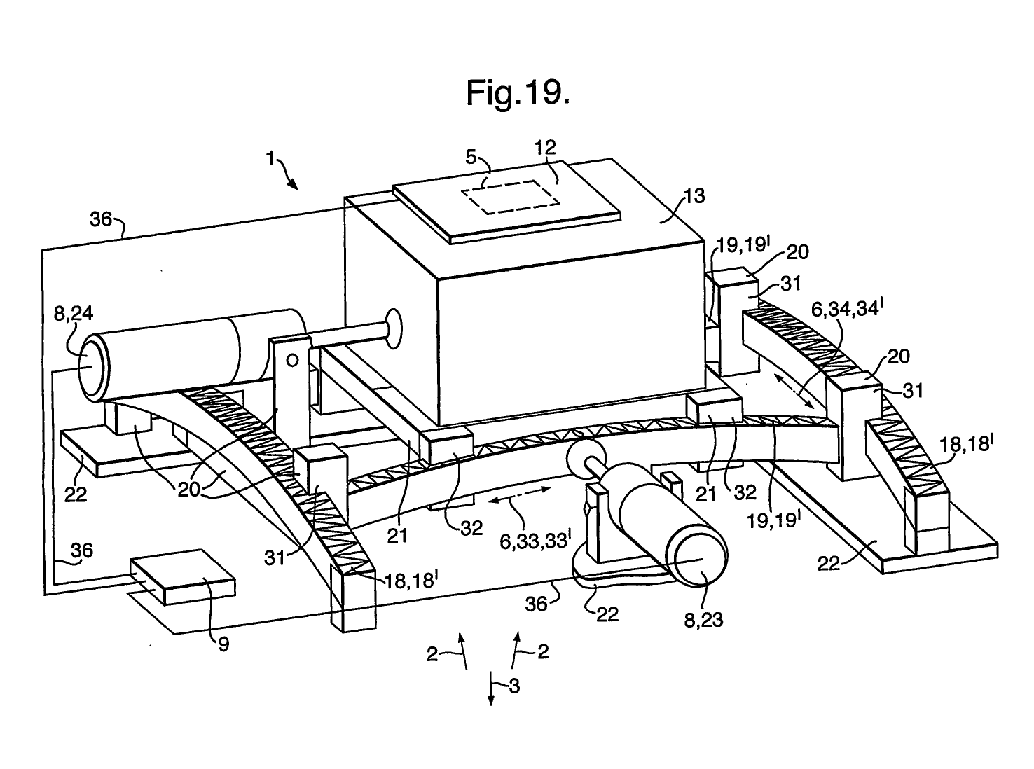

FIG. 19 shows a perspective view of an apparatus in which a VApC according to an embodiment of the present invention is mounted;

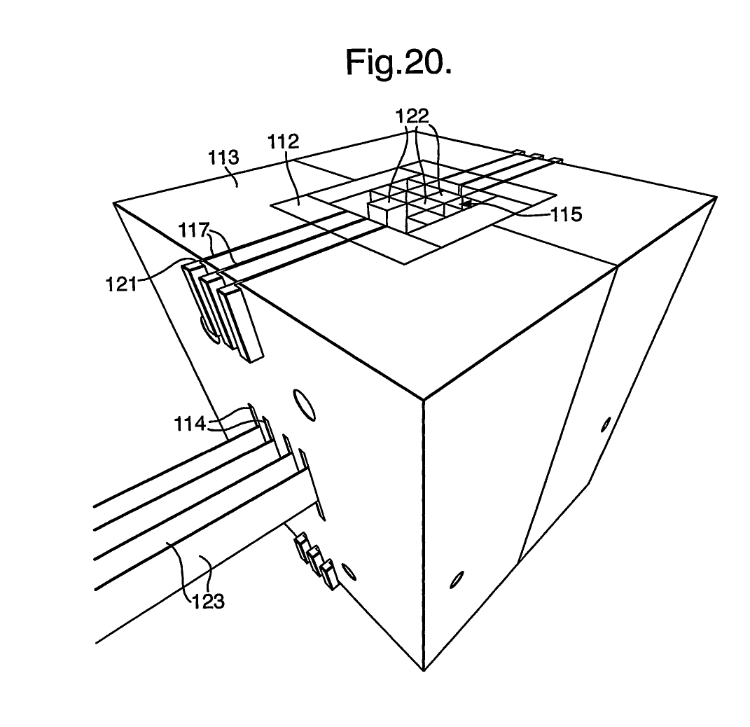

FIG. 20 shows a perspective view of a VApC according to another embodiment of the present invention.

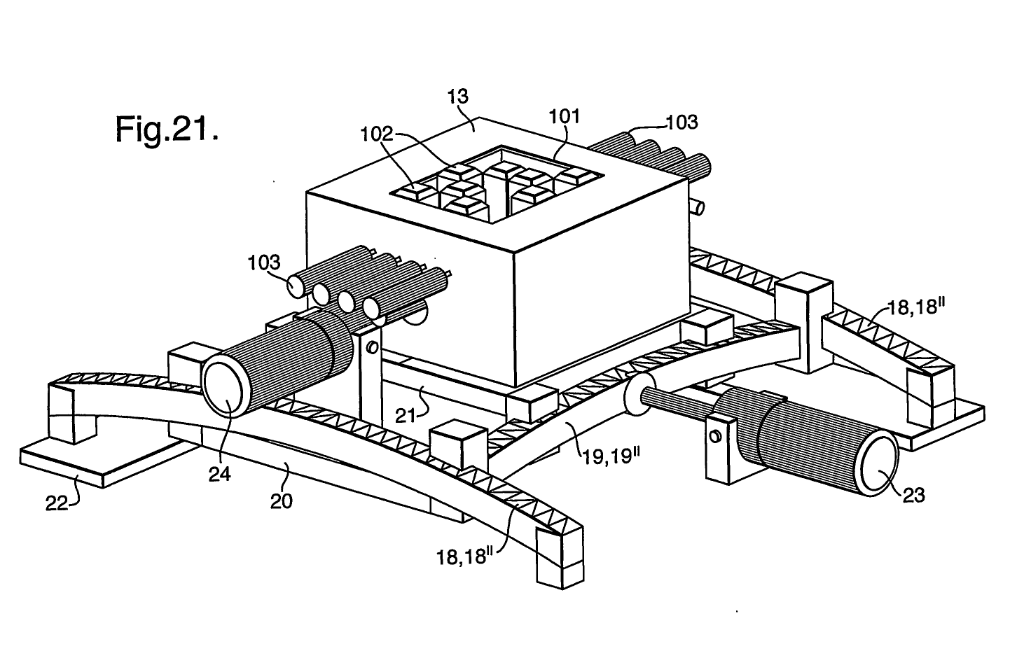

FIG. 21 shows a perspective view of an apparatus in which a VApC according to another embodiment of the present invention is mounted;

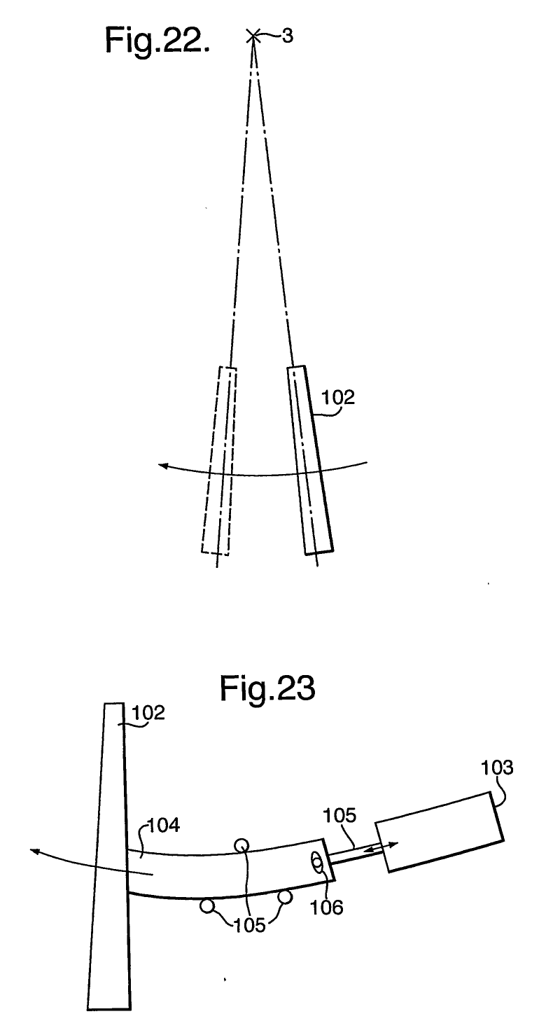

FIG. 22 shows a side view of a single beam attenuator for the VApC of FIG. 21; and

FIG. 23 shows schematically a mechanism for moving the single beam attenuator of FIG. 22.

A first embodiment of a collimator according to the present invention is shown in FIGS. 1a and 1b. Such a collimator will generally be referred to as a variable-aperture collimator or VApC. The VApC shown in FIGS. 1a and 1b will be called VApC1 for reference purposes.

The window of this VApC1 comprises an array of 3 columns of 5 rows of bixels. The surrounding, un-illustrated area is a surround which blocks the passage of radiation outside the collimator window. This surround may be made from tungsten or similar material. A similar surround is assumed for all the VApCs illustrated in FIGS. 1 to 5, 7 and 8.

In their rest or parked position six radiation attenuators, also called single-bixel attenuators (SBAs), reside in the 1st and 5th row. A simple push-and-lock mechanism may advance each of these SBAs (2 for each column) into any of the other three vacant spaces in the same column (and at the same time creating another open bixel space in the position they are moved from). One possible resulting pattern of the attenuators is shown in FIG. 1b, each of the columns therein showing at least one blocked bixel which is sandwiched between a pair of open bixels. Other methods of moving the SBAs along the columns are possible including rods driven by solenoids, rack mechanisms, hydraulics, electric drive units or pneumatics.

Thus for each column there are

$$\frac{5 !}{3 ! 2 !} = 10$$

ways to position the 3 open spaces (choosing 2 (or 3) positions from 5 where order is not important). There are thus 10³ ways to arrange such a 5-row by 3-column aperture, containing 9 open bixels and 6 closed bixels, where each pair of SBAs are constrained to move in a single column. Provided that the jaws to the left and/or right of the VApC as illustrated can be moved to cover either one or two columns of the collimator window, thereby adjusting the dimensions of the window, there will be 10³+10²+10=1110 possible apertures.

A brief description is made of a highly simplified decomposition or “stripping” method using a jaws only (JO) technique as illustrated in FIG. 6. This is provided so that the terms in the following description of the decomposition method using a VApC can be understood.

IMRT planning systems generate a “map” of varying MUs per bixel, such as that shown as the “Starting IMB” in FIG. 6. This is the IMB that is to be delivered, e.g. to the patient. To break down this starting IMB using the JO technique, the largest rectangular group of bixels with a common amount of fluence (number of MUs) is selected as shown as the “1st component” in FIG. 6. This maximum amount of common fluence is then “stripped off” as the first component, leaving a residual IMB. This technique is repeatedly applied to the residual IMB from each “stripping” action until the residual IMB is empty, as shown in FIG. 6.

From this illustration, it can be seen that two particular parameters of the stripping process are important from a practical perspective: the number of components or stripping actions that are necessary for the decomposition (M); and the total number of MUs that are stripped (and therefore will need to be delivered) in order to carry out the decomposition (MU).

The first of these parameters (M) affects the physical practicality of delivering the IMB using the decomposition, since each component requires some form of re-positioning of the jaws/mask/collimator. The second of these parameters (MU) represents the total amount of radiation that the source will need to deliver to create the starting IMB, and therefore the energy “cost” of the decomposition. It is therefore desirable that both these parameters are kept as low as possible by any IMRT technique that involves decomposing the beam in this way.

The general decomposition method using a VApC starts with the N×N IMB it is desired to decompose. The stripping algorithm then tries all positions of the VApC (in this case VApC1) within the IMB space and, at each position, all the possible apertures. The aperture with the largest number of irradiated bixels is selected. If more than one satisfies this condition, then that with the largest sum of MUs across all irradiated bixels is selected. This is a far-from trivial computational task since, including the possibility that only part of the VApC1 is within the IMB space, it is required to test (N+2)×(N+4)×1110 options for each component strip (using VApC1). When the process is activated for a large number of random IMBs (e.g. 1000 cases at each Iₚ value) the computational complexity becomes apparent. Nevertheless, when modelled on a COMPAQ Alphastation 250/4 266, this can be achieved within an hour for all 1000 cases on all Iₚ values in the range from 3 to 15. Thus the complexity is not a limitation.

At each cycle of component strip, the minimum fluence in the linked set of bixels is found and that is stripped off to create a residual IMB. The process cycles until the VApC1 can sweep no more because the remaining bixels are too isolated. At this stage a final sweep of a single open bixel is required. Finally the residual IMB is empty and the procedure for delivering the original IMB using the VApC can be determined. The outcome is a set of components, specifying the six locations of the SBAs; the jaw positions (if used); the MUs per component; and the position of the VApC in the overall IMB.

A second embodiment of a VApC according to the present invention (VApC2) is shown in FIGS. 2a and 2b. This VApC2 comprises 4 columns of 5 rows. In their rest or parked position eight single-bixel attenuators (SBAs) reside in the 1st and 5th row. There are thus 10⁴ ways to create a 5-row by 4-column aperture having 12 open bixels and 8 closed bixels, where each pair of SBAs are constrained to move in a single column. One possible arrangement of the SBAs in this embodiment is shown in FIG. 2b. In three of the columns, single blocked bixels are sandwiched between a pair of open bixels. In the right hand column, a line of two blocked bixels is sandwiched between a pair of open bixels.

Provided the jaws can also be used to cover either one, two or three columns there will be 10⁴+10³+10²+10=11110 possible apertures. Decompositions for VApC2 were performed by the algorithm described above in relation to the VApC1. It would be expected that the performance would be an improvement on the use of VApC1 given the larger number of options available for the linked isolated bixel shapes.

A third type of VApC (VApC3), shown in FIGS. 3a and 3b, has 3 columns of 7 rows. Twelve SBAs (i.e. twice the number as in VApC1) reside in parked position in rows 1, 2, 6 and 7. These can be moved to any of the other rows such that there are 4 SBAs located in each column in any one configuration. One possible arrangement of the SBAs in this embodiment is shown in FIG. 3b. In two of the columns, single blocked bixels are sandwiched between a pair of open bixels. In two of the columns, lines of two or three blocked bixels are sandwiched between a pair of open bixels.

There are therefore

$$\frac{7 !}{4 ! 3 !} = 35$$

ways to arrange the SBAs in any one column and so 35³+35²+35 =44135 possible aperture shapes. Decomposition was performed for VApC3 followed by a subsequent “mop-up” with a single open-bixel aperture.

A fourth type of VApC (VApC4), shown in FIGS. 4a and 4b, has 4 columns of 7 rows. Eight SBAs reside in parked position in rows 1 and 7. These can be moved to any of the other rows. One possible arrangement of the SBAs in this embodiment is shown in FIG. 4b. In all of the columns, single blocked bixels are sandwiched between a pair of open bixels.

There are

$$\frac{7 !}{5 ! 2 !} = 21$$

ways to arrange the SBAs in any one column and so 21⁴+21³+21²+21=204204 possible aperture shapes (assuming use of jaws). Decomposition was performed for VApC4 followed by a subsequent “mop-up” with a single open-bixel aperture.

A fifth type of VApC (VApC5), shown in FIGS. 5a and 5b, has 4 columns of 9 rows. Eight SBAs reside in parked position in rows 1 and 9. These can be moved to any of the other rows. One possible arrangement of the SBAs in this embodiment is shown in FIG. 5b. In all of the columns, single blocked bixels are sandwiched between a pair of open bixels.

There are

$$\frac{9 !}{7 ! 2 !} = 36$$

ways to arrange the SBAs in any one column and so 36⁴+36³+36²+36=1727604 possible aperture shapes. Decomposition was performed for VApC5 followed by a subsequent “mop-up” with a single open-bixel aperture.

A hybrid decomposition was also performed, beginning with VApC4 and, when this could form no more components, switching to VApC2, finally mopping up with single open bixels. This hybrid VApC is shown in FIG. 7, and can be accomplished by reducing the aperture size of the VApC4 in the direction shown to reduce the number of rows from 7 to 5.

It is apparent from FIG. 7 that one can extend this concept to larger-area VApCs with different numbers of rows. We considered the situation where the number of columns was restricted to four. Then each VApC can be designed with R rows and one attenuating element parked at each end of the 4 columns and capable of being moved into any of the other (R−2) rows. The number of possible configurations in any one column is then

$$C = \frac{R !}{( R - 2 ) ! \cdot 2 !} = \frac{R \times ( R - 1 )}{2} .$$

The number of configuration options altogether is then C⁴+C³+C²+C. Other VApCs were considered with R varying from 3, 4, 5 . . . 15, some of which are of course those described above and shown in FIGS. 1 to 6.

Hybrid decompositions were then systematically modelled starting with the hybrid which effectively had the combination of VApCs with 5, 4 and 3 rows (no single mop-up is necessary in the case of a hybrid VApC capable of having 3 rows as this is taken care of by the VApC with 3 rows when the jaws are used). The 2nd hybrid effectively had the four VApCs with 6, 5, 4 and 3 rows. The 3rd hybrid had the five VApCs with 7, 6, 5, 4 and 3 rows and so on until the last hybrid considered had all the possible VApCs with 15, 14, . . . 3 rows as shown conceptually in FIG. 8.

Finally two more possibilities were tested: to decompose IMBs using just the full-width VApC (or hybrid VApC), i.e. without use of jaws to allow reduction in the number of columns in the aperture; and allowing the VApC (or hybrid VApC) to rotate by 90° with respect to the IMB. This, for any choice of VApC or hybrid VApCs leads to four options. These are decomposition with or without jaws and with or without rotation. The same IMB decomposition problems were studied for these options.

The results of using the various VApCs described above with the stripping algorithm described above in a benchmark simulation of 1000 15×15 bixel IMBs with fluence (or MU) values for each bixel being integers between 3 and Iₚ are shown in FIGS. 9-11. These Figures show either the mean number of components <M> required to decompose the IMB, or the mean number of monitor units <MU> required to decompose the IMB, for each value of Iₚ.

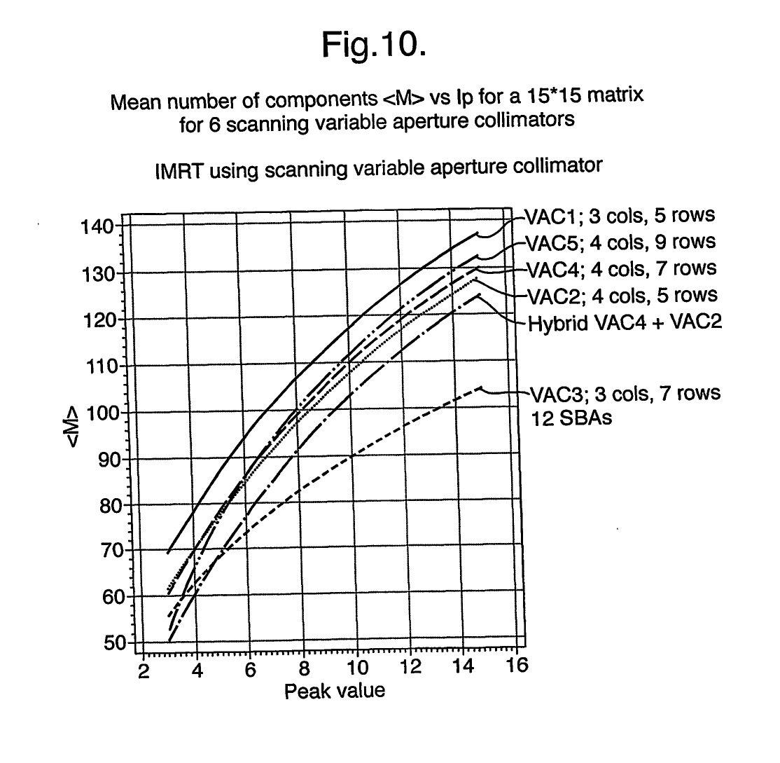

FIGS. 9 and 10 show <MU> and <M> respectively for the different forms of simple VApC described in the above embodiments and the hybrid having a combination of VApC4 and VApC2.

We note that VApC5 alone becomes a worse option than VApC4 alone which in turn is worse than VApC3 alone. This is because, whilst larger-area VApCs can open wide-area segments, they are less good at connecting isolated bixel islands.

It turns out that the hybrid stripping has the best MU performance for all Iₚ, and at least up to Iₚ=5 the best performance in minimising the mean number of components.

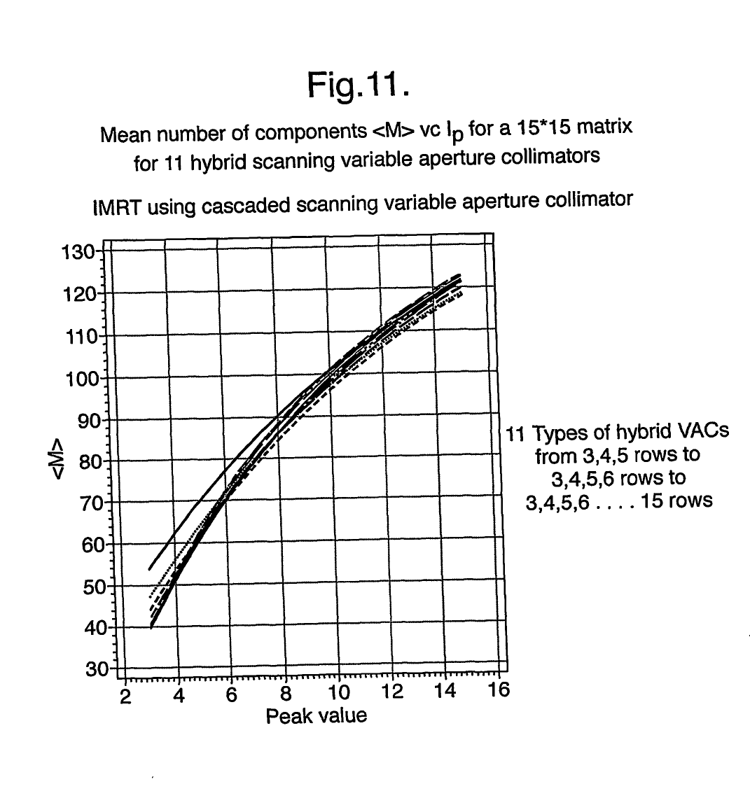

FIG. 11 shows the mean number <M> of field components required to strip a 15×15 IMB with random integer fluences (MUs) between 3 and Iₚ in the bixels using the various forms of the hybrid VApCs having from 5 to 15 starting rows.

Interestingly the gradients of the curves change so that what is best at one particular Iₚ is not always best at other values of Iₚ. The detailed dependences on the form of each VApC hybrid can only be appreciated by studying the tables (not shown) of performance of each of the 11 options (VApCs with 3,4,5 rows through to VApCs with 3,4,5 . . . 15 rows) with respect to each of the 13 peak values Iₚ.

In FIGS. 9 to 11, the jaws of the collimator were used to reduce the number of columns in the VApC as the decomposition progressed. FIGS. 12 and 13 show the effects of not using the jaws in this way, and of allowing the VApC to be rotated through 90° during the decomposition, both when the jaws were used and when they were not.

From FIGS. 12 and 13, it can be seen that the ranking for these four options is the same for both VApC2 and for the use of the full hybrid VApC. It can also be seen that curves for jaws with no rotation, and rotation with no jaws are very close together and interestingly cross over. This shows that the use of rotation without jaws is very similar to the use of jaws without rotation. The choice between these options may therefore be made on the basis of engineering practicalities. It can also be seen that the gradients are different for the use of VApC2 than for the use of the full hybrid VApC. Finally it is noted that, whilst the use of the full hybrid VApC is clearly better when there is no rotation allowed and no jaws, the addition of the option to use rotation makes VApC2 with jaws have better performance than the hybrid VApCs with jaws (but no rotation) for large Iₚ values whilst vice-versa for low Iₚ values.

By comparing all these results with FIGS. 16 and 17 we see the enormous improvement over the use of the jaws-only (JO) technique, and comparable performance with the J+M technique using 2 or 4 individually relocatable SBAs.

The comparisons of <M> and <MU> are useful, but only address two aspects of delivery of an IMB. The use of VApCs offers improvements in the practicalities of delivering the components of the decomposed IMB, both in terms of engineering and time taken.

Furthermore it has been discovered that complete stripping of an IMB may not be entirely necessary. The result of stripping algorithm described above is the formation of widely separated islands of residual fluence that are difficult or impossible to couple together, necessitating many passes of a single open bixel to sweep up the residuals at the end. The same decompositions were made with stripping stopped when there was just 2% residual fluence. This is expected (and shown) to lead to the need for fewer components and fewer monitor units.

The comparative results of stopping the stripping algorithm when only 2% residual fluence remained are shown in FIGS. 14 and 15. In these Figures, the lines marked A, B and C are the values for VApC1, VApC2 and VApC3 respectively with full decomposition performed; the lines marked A′, B′ and C′ are the values for VApC1, VApC2 and VApC3 respectively when decomposition was stopped with 2% residual fluence remaining.

It is interesting to compare the results obtained using the various VApCs described with the results which can be obtained using a conventional MLC. There are many algorithms for decomposing any IMB using a standard MLC. Xia and Verhey (1998 Medical Physics 25, 1424-1434) compared the Bortfeld-Boyer algorithm, the Galvin algorithm and their own (then new) power-of-two fluence decomposition algorithm for randomly generated matrices of size 15×15 and peak values of Iₚ=3, 4 . . . 15, as used in relation to the embodiments above. The results are shown in FIG. 8 of that paper. The MLC results depend on whether interdigitation constraints are obeyed or ignored. The Xia and Verhey paper showed that the Bortfeld-Boyer algorithm always led to the generation of the minimum <MU> whatever the value of Iₚ. For example, with Iₚ=15, <MU> is about 60 when constraints are not applied. Conversely their own algorithm generated the least <M> at all Iₚ (e.g. typically about 20 at Iₚ=15). Later studies by Que (1999 Medical Physics 26, (11), 2390-2396) introduced further competing algorithms.

These MLC-based techniques will lead to the requirement for fewer segments and fewer MUs than the non-MLC, VApC, techniques. However, not all IMRT centres have linacs fitted with an MLC. Centres with a linac without an MLC or which deliver radiotherapy using a cobalt-60 machine (which has no MLC) would benefit from alternative techniques to deliver IMRT. This is particularly useful in countries where the use of linacs is less common.

One implementation using a VApC includes locating it in a double-cradle arrangement so that whatever position is taken up by the VApC it is focused to the radiation source. The elements (SBAs) of the VApC itself are preferably wedge-shaped, tapered to the source.

A brief description of a cradle of this type is made in relation to FIGS. 18 to 19 below. It consists of a collimator housing which can be moved two-dimensionally upon the surface of sphere of which the center is located at the beam focus. By this means any pattern of scanning pathways can be generated using only two driving motors. When moving the collimator housing, the jaws are moved at the same time in such a way that the necessary shielding outside the collimator housing is always obtained. In other words, the jaws overtake the function of shielding curtains. This collimator housing can serve to include one of the VApCs as described above.

FIG. 18 is a diagram showing the underlying principle of the type of apparatus in which a VApC may be used. As shown, the VApC aperture 5 delimits rays 2 coming from a radiation source 3 so that said rays act upon an area 37 which is considerably smaller than the area 26 over which irradiation is to be effected. The aperture 5 is situated in a shielding block 13 which is displaceable on a path in the shape of a spherical surface 6 so that the rays 2′ which pass through the aperture 5 scan the area 26 to be irradiated, by means of a corresponding drive 8 which is not illustrated here, and thus act upon said area with the desired irradiation.

The irradiation area 26 thus corresponds to the shape of the treatment object 4 in the direction of irradiation of the irradiation which is presently effected. This is explained in more detail below. The displacement of the shielding block 13 with the aperture 5 on the path in the shape of a spherical surface 6 occurs by scanning movements 33, 33′ with respect to the spherical surface being made in the x direction and by scanning movements 34, 34′ with respect to the spherical surface being made in the y direction. In the course of this procedure, the aperture 5 is aligned so that its centre line 7 points towards the radiation source 3. Furthermore, the boundaries 10 of the aperture 5 are aligned so that they taper in the direction of the beam path 2, 2′, so that the entire thickness of the shielding block 13 is always available for shielding and there is no incomplete shadow due to insufficient shielding. The shielding block 13 then has to be guided whilst this alignment of the aperture 5 is maintained. An embodiment of a guide system of this type is shown in FIG. 19, although other types of guidance are also possible, of course.

FIG. 18 also shows that the rays 2′ coming from the radiation source 3 are delimited by a pre-collimator 35, where the aperture of this pre-collimator 35 is dimensioned so that it shields all the regions situated outside the shielding provided by the shielding block 13, so that in each of its possible positions the shielding block 13 provides a shield from the rays 2 passing through the pre-collimator except for the rays 2′, which pass through its own aperture 5. The aperture of the pre-collimator 35 could also, of course, be variable or displaceable.

FIG. 19 is a perspective view of an apparatus 1 in which a VApC is mounted. In this illustration, the radiation source 3 is situated underneath the apparatus which is illustrated and the rays 2 impinge on the VApC from this direction. The radiation source 3 and the pre-collimator 35 have been omitted for the sake of simplicity.

The embodiment illustrates how a drive 8 can be created. So as to be able to execute movements on the path in the shape of a spherical surface 6, a first sliding rail 18 is first of all disposed in a collimator housing 22, only a fragment of which is illustrated. This first sliding rail 18 consists of a pair of rails 18′ and 18″ which are arcuate in shape, so that the centres of these two arcs are situated on an axis which passes through the approximately point-like radiation source 3. A first displaceable sliding carriage 20 is disposed on this first sliding rail 18, and comprises bearings 31 which run on the first sliding rail 18 and a second pair of rails 19′ and 19″ which forms a second sliding rail 19 which extends perpendicularly to the first sliding rail 18. The pair of rails 19′ and 19″ of the second sliding rail 19 are also of arcuate construction, and the centres of these arcs are also situated on an axis which passes through the radiation source 3. A second sliding carriage 21, which can be displaced by means of bearings 32, is disposed on the second sliding rail 19. The shielding block 13, in which the VApC aperture 5 is situated, is mounted on this second sliding carriage 21.

The two sliding carriages 20 and 21 make it possible to effect a displacement on the path in the shape of a spherical surface 6, so that scanning movements 33, 33′ can be executed in the x direction and scanning movements 34, 34′ can be executed in the y direction. A drive 23 for the first sliding carriage 20 is employed for this purpose and is disposed on the collimator housing 22. This drive serves to execute the scanning movements 34, 34′. A drive 24 for the second sliding carriage 21 is disposed on the first sliding carriage 20, and is employed for displacement in the x direction, namely for executing scanning movements 33 and 33′. Here also, the x and y directions do not relate to a planar surface, but relate to the spherical surface of the path in the shape of a spherical surface 6.

In order to effect scanning movements 33, 33′, 34, 34′, which are executed so that an irradiation area 26 can be acted upon by the predetermined irradiation, a control means 9 is provided which is connected by connections 36 to the drives 23 and 24.

The VApC is illustrated only schematically in FIG. 19. Typically, however, it comprises a device 12 mounted in the shielding block 13 and having a plurality of SBAs (not shown) which are independently movable within a window through the device. When the window is viewed from the radiation source it defines a two-dimensional bixel array, and the SBAs are only movable along columns or rows of this array. The control means 9 is connected to the SBAs and controls their movements. Typically also, the shape and size of the window through the device 12 is alterable by movable jaws (not shown) which form the edges of the window and are controlled by the control means 9. Thus, together the jaws and SBAs define the VApC aperture 5 which is aligned towards the radiation source 3 as explained above with reference to FIG. 18.

One embodiment of a VApC of the present invention is shown in perspective view in FIG. 20. This VApC comprises a tungsten framework 112 made of Wolfmet™ HE395 mounted within an outer aluminium framework 113. Whilst pure tungsten is best suited to attenuation of high-energy X-rays, it is very brittle and thus hard to work. Consequently tungsten alloys such as Wolfmet™ HE395 are used in practice.

The tungsten framework 112 has a width of at least 5 mm and together with the jaws of the device (not shown) fulfils the task of excluding radiation outside the beam window 115. Experience from other collimators shows that a tungsten thickness (i.e. height of the collimator as shown) of 7-9 cm causes sufficient attenuation of radiation in the energy region of 6 MeV.

The inner surfaces of the tungsten framework 112 are adapted to the beam divergence so that they have the appropriate inclination, the exact dimensions of the interior and exterior of the tungsten framework are defined by the selected field size in consideration of standard intercept theorems and the Source Collimator Distance (SCD—typically of the order of 520 mm) and the Source Isocentre Distance (SID—typically of the order of 1000 mm).

Perpendicular slots 114 are provided halfway up the two frameworks and serve as passages for the SBA guidance. These slots are also curved in accordance with the intercept theorems.

At the upper and lower ends of the VApC as viewed, further thin slots 117 are provided which accommodate auxiliary guidance 121 which assist in keeping the SBAs aligned within the collimator.

In the collimator shown the array has four columns and five rows, and in each column two independently movable SBAs 122 are situated, giving 8 SBAs in total. Each SBA 122 is made from tungsten and is of the same height as the tungsten framework 112. The SBAs are shaped to taper towards the radiation source in accordance with the intercept theorems and thus provide a constant penumbra at the isocentre in all positions in the array.

An arc shaped guidance 123 is connected at one end to each of the SBAs 122, and passes through one of the perpendicular slots 114 in the tungsten and aluminium frameworks.

To achieve better sliding qualities, a bronze guide bearing (not shown) is located in each of the perpendicular slots 114. This guide bearing has a milled slot describing an arc shaped course along the bearing corresponding to the arc shape of each guidance 123.

FIG. 21 is a perspective view of another double cradle apparatus in which a VApC is mounted. The apparatus has a control means and connections like the apparatus of FIG. 19, but to avoid repetition these are omitted in FIG. 21.

The VApc of FIG. 21 is integrated with the shielding block 13. A window 101 through the block defines a two-dimensional bixel array when the window is viewed from the radiation source. The array has four columns and five rows and in each column two independently movable SBAs 102 are situated.

A drive 103 is provided for each SBA. The drives are connected to the control means and under its control move the SBAs along their respective columns. The control means and drives are not able to move the SBAs outside their respective columns, but changing the positions of the SBAs within their columns nonetheless leads to different bixel patterns. Effectively, each pattern formed within the window produces a different VApC aperture. Thus changing the bixel pattern between irradiations allows a predetermined pattern of radiation intensities to be delivered through the VApC.

FIG. 22 shows a side view of an SBA 102 for the VApC of FIG. 21 (or indeed FIG. 19 or 20). The SBA is an elongate, wedge-shaped member which extends and tapers towards the radiation source 3. Having this shape, when the SBA is moved along the circular arc indicated by the arrow to another position (indicated by dashed lines), the penumbra formed by the SBA does not vary. Viewed from the radiation source, the circular arc projects as a straight line along a column of the bixel array. Thus moving the SBA along the arc is equivalent to moving the SBA along its column.

FIG. 23 shows schematically a mechanism for moving the SBA of FIG. 22 along the circular arc. An elongate curved metal plate 104 is rigidly attached at one end to the SBA 102. The plate is relatively thin so that it does not produce a significant penumbra when irradiated by the radiation source. The plate 104 is held between three guide pins 105 which support the SBA and the plate while still allowing guided movement along the arc. The other end of the plate is linked to a push rod 105 of the drive 103 for the SBA. The linkage 106 between the plate and the push rod is slightly loose so that guided movement of the plate along the curved arc (indicated by the single headed arrow) can be actuated by straight line movement (indicated by the double headed arrow) of the push rod.

1. A method of intensity-modulating a beam of radiation from a radiation source, the method including the steps of:
a) providing a collimator having a window which allows the passage of radiation from the radiation source and which defines when viewed from the radiation source a two-dimensional array of bixels, the collimator further having a plurality of independently movable radiation attenuators which are constrained to move along columns or rows of the array to block selected bixels thereof, whereby, within each column or row along which the attenuators are movable, at least one arrangement of the respective attenuator(s) results in a pair of open bixels sandwiching a blocked bixel or line of blocked bixels;
b) positioning the collimator in the path of the radiation source; and
c) repeatedly:
positioning the radiation attenuators within the window to form at each repeat a different pattern of bixels; and
irradiating the collimator;
until a predetermined pattern of radiation intensities for the beam of radiation has been delivered through the collimator.

2. A method according to claim 1, wherein the collimator is repositioned one or more times during the performance of step c).

3. A method according to claim 1, wherein at least one of the size and position of the window is adjusted one or more times during the performance of step c).

4. A method according to any one of claims 1-3 or 11, wherein the collimator is rotated about an axis perpendicular to the window one or more times during the performance of step c).

5. A method of radiotherapy treatment performed on a human or animal body comprising subjecting the body to an intensity-modulated beam of radiation formed according to any one of claims 1-3 or 11.

6. An apparatus for producing an intensity-modulated beam of radiation including:
a radiation source;
a collimator having a window which allows the passage of radiation from the radiation source and defines when viewed from the radiation source a two-dimensional array of bixels, the collimator further having a plurality of independently movable radiation attenuators which are constrained to move along columns or rows of the array to block selected bixels thereof, whereby, within each column or row along which the attenuators are movable, at least one arrangement of the respective attenuator(s) results in a pair of open bixels sandwiching a blocked bixel or line of blocked bixels; and
a control system for controlling the position of the radiation attenuators within the window of the collimator.

7. An apparatus according to claim 6, wherein the size of at least one of the collimator window and position of the collimator window relative to the radiation source are adjustable, and the control system also controls at least one of the size and/or position of the collimator window.

8. An apparatus according to claim 6 wherein the collimator is rotatable about an axis normal to the window, and the control system also controls the rotation of the collimator.

9. An apparatus according to any one of claims 6 to 8 or 13, wherein the control system further controls the intensity of the beam emitted from the radiation source.

10. (canceled)

11. A method according to claim 2, wherein at least one of the size and position of the window is adjusted one or more times during the performance of step c).

12. A method of radiotherapy treatment performed on a human or animal body comprising subjecting the body to an intensity-modulated beam of radiation formed according to claim 11.

13. A method of radiotherapy treatment performed on a human or animal body comprising subjecting the body to an intensity-modulated beam of radiation formed according to claim 4.

14. An apparatus according to claim 7 wherein the collimator is rotatable about an axis normal to the window, and the control system also controls the rotation of the collimator.

15. A collimator, comprising a window which allows the passage of radiation from a radiation source and defines when viewed from the radiation source a two-dimensional array of bixels, the collimator further having a plurality of independently movable radiation attenuators which are constrained to move along columns or rows of the array to block selected bixels thereof, whereby, within each column or row along which the attenuators are movable, at least one arrangement of the respective attenuator(s) results in a pair of open bixels sandwiching a blocked bixel or line of blocked bixels.

16. A collimator according to claim 15, wherein at least one of the size of the collimator window and position of the collimator window relative to the radiation source are adjustable.

17. A collimator according to claim 15 or 16 wherein the collimator is rotatable about an axis normal to the window.
